(19) United States
(12) Patent Application Publication
(10) Pub. No.: US 2006/0217431 A1
(43) Pub. Date: Sep. 28, 2006

(54) Composition comprising a combined thromboxane receptor antagonist and thromboxane synthase inhibitor and a COX-2 inhibitor

(75) Inventors: Juergen Daemmgen, Ochsenhausen (DE); Joanne van Ryn, Warthausen (DE); Brian Guth, Warthausen (DE); Michel Pairet, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/276,972

(22) Filed: Mar. 20, 2006

(30) Foreign Application Priority Data
Mar. 23, 2005 (EP) 05006325

(51) Int. Cl. A61K 31/557; A61K 31/365; A61K 31/415

(52) U.S. Cl. 514/406; 514/471; 514/573

(57) ABSTRACT

The invention relates to a pharmaceutical composition comprising a combined thromboxane receptor antagonist and thromboxane synthase inhibitor and a COX-2 inhibitor. In addition a method of treating cyclooxygenase dependent disorders, including inflammation, pain and/or rheumatic diseases, and/or neoplasia is described.

This invention relates to a pharmaceutical composition comprising a combined thromboxane receptor antagonist and thromboxane synthase inhibitor and a cyclooxygenase-2 (COX-2) inhibitor. Furthermore this invention relates to a pharmaceutical dosage form comprising such a composition. A further objective of this invention is related to the use of the composition and the pharmaceutical dosage form. In addition this invention relates to the use of a combined thromboxane receptor antagonist and thromboxane synthase inhibitor and a COX-2 inhibitor for the manufacture of such a pharmaceutical dosage form. Furthermore, this inventions relates to a method of treating cyclooxygenase dependent disorders, including inflammation, pain and/or rheumatic diseases, and/or neoplasia in a patient in need of such treatment, which comprises administering to the patient a combination of a combined thromboxane receptor antagonist and thromboxane synthase inhibitor and a COX-2 inhibitor.

### BACKGROUND OF THE INVENTION

Non-steroidal anti-inflammatory drugs (NSAIDs) are among the most commonly taken drugs for inflammatory conditions and pain. NSAIDs act by inhibiting cyclooxygenase (COX), the enzyme responsible for generation of prostaglandins that not only contribute to pain and inflammation but are cytoprotective. Thus because NSAIDs indiscriminately inhibit both isoforms of COX (constitutive COX-1 responsible for cytoprotective effects and inducible COX-2 responsible for inflammatory effects), they are associated with increased toxicities such as gastric mucosal erosions and ulcers and renal toxicity. Hence the search for specific inhibitors of COX-2 which would be as effective as traditional NSAIDs in terms of anti-inflammatory activity and pain relief but would be associated with fewer adverse effects.

The chronic use of aspirin, a COX-1 inhibitor, has also been associated with a reduced incidence of cardiovascular disease and many people presently take low doses of aspirin on a daily basis to reduce their risk of stroke and thromboembolism. Aspirin exerts this effect by inhibiting cyclooxygenase-1 (COX-1), an enzyme that contributes to the production of thromboxane, particularly in platelets. While all NSAIDs inhibit COX-2, they inhibit COX-1 to varying degrees.

More recently it has been determined clinically that the selective inhibition of COX-2, with no COX-1 inhibition at therapeutic doses, is associated with an elevated risk for adverse cardiovascular events. It appears that COX-2 in the vascular wall is important in the production of prostacyclin, which dilates blood vessels and prevents platelets from clumping together. The loss of this activity coupled with unchecked thromboxane production (via COX-1) may result in a prothrombotic state and significantly increase the risk of cardiovascular events.

The international application WO 01/87343 is related to a combination therapy using a COX-2 selective inhibitor and a thromboxane inhibitor, in particular in patients who are at risk of developing thromboembolic events.

The international application WO 2004/004776 describes a pharmaceutical combination of a thromboxane A2 receptor antagonist and a COX-2 inhibitor for the treatment of inflammation, pain and cardiovascular disorders.

A pharmaceutical combination of a thromboxane A2 receptor antagonist and a COX-1 inhibitor is described in the international application WO 2005/016334. It is proposed that this combination is useful in the treatment of peripheral arterial disease, arterial or venous thrombosis, unstable angina, transient ischemic attacks and hypertension.

### OBJECTIVE OF THE PRESENT INVENTION

The aim of the present invention is to further improve the safety profile of COX-2 inhibitors with respect to possible adverse side effects, in particular thrombotic and cardiovascular events, and to provide a pharmaceutical composition and a pharmaceutical dosage form comprising a COX-2 inhibitor.

### BRIEF SUMMARY OF THE INVENTION

This invention relates to new pharmaceutical compositions and pharmaceutical dosage forms comprising a combined thromboxane receptor antagonist and thromboxane synthase inhibitor and a COX-2 inhibitor.

The present invention also relates to a method of treating cyclooxygenase dependent disorders, including inflammation, pain and/or rheumatic diseases, and/or neoplasia in a patient in need of such treatment, which comprises administering to the patient a combination of a combined thromboxane receptor antagonist and thromboxane synthase inhibitor and a COX-2 inhibitor.

The combination of a combined thromboxane receptor antagonist and thromboxane synthase inhibitor and a COX-2 inhibitor improves the safety profile of the COX-2 inhibitor. In particular the risk of thromboembolic and/or cardiovascular events, such as myocardial infarction, intermediate coronary syndrome or sudden cardiac death due to a selective inhibition of COX-2 can be reduced or even prevented. Therefore this finding allows to continue to take advantage of the therapeutic benefits of COX-2 inhibitors which otherwise might increase the risk of thromboembolic and/or cardiovascular events when taken alone.

Without being bound to any theory, in the following the proposed mechanistic aspects underlying the present invention and the resulting advantages are described.

The selective blockade of thromboxane receptors does not interfere with the production and metabolism of arachidonic acid in activated platelets, but it does prevent the activation of platelets by the binding of thromboxane or endoperoxides (prostaglandin intermediates) to the thromboxane receptor. Thromboxane/endoperoxide receptor blockade also prevents the vasoconstrictive action of thromboxane on smooth muscle cells. In addition, the selective inhibition of thromboxane synthase prevents the conversion of prostaglandin endoperoxide intermediates (PGH₂, PGG₂) to thromboxane.

The combination according to this invention of receptor blockade and synthase inhibitor has the advantage of inhibiting platelet function by blocking the thromboxane receptor and allowing the build-up of other arachidonic acid metabolites in the platelet, i.e. they can no longer be converted into thromboxane, so they build up within the cell. Thus, in the setting of platelet activation as would occur locally at a vascular injury site, the platelet-produced endoperoxides can be taken up by other cells (smooth muscle cells, endothelial cells and white blood cells). These endoperoxides can then be used as the substrate for prostacyclin (PGI₂) production and other antithrombotic prostanoids. Since COX-2 inhibition in the vessel wall reduces the amount of prostacyclin produced by preventing PGH₂ and PGG₂ formation, in this setting the platelets could supply the missing endoperoxides and the vessel wall (endothelium, smooth muscle cells) could produce prostacyclin even in the presence of a COX-2 inhibitor. Essentially, the platelet would act as the source of endoperoxide intermediates (substrate) for use by the vessel wall to produce prostacyclin.

Additionally, not only the end product prostacyclin, but the conversion of the endoperoxides to E-type prostaglandins could help to reduce thrombus formation due to their vasodilatory action. Finally, the leukocyte inhibiting activity of E-type prostaglandins could also help to reduce any ischemic damage which may occur distal to an obstructive thrombus.

This mechanism would not function with only thromboxane receptor blockade, as this would not block the production of thromboxane within platelets and there would be no build up of intermediate prostaglandins and no substrate for prostacyclin in the vessel wall. In addition, with only thromboxane synthase inhibition there may be some build up of intermediate prostaglandins but platelet activation and clumping is not inhibited because the receptor is not blocked.

Therefore the invention further relates to the use of a combined thromboxane receptor antagonist and thromboxane synthase inhibitor for the manufacture of a medicament for the treatment or prophylaxis of adverse events, in particular of thromboembolic and/or cardiovascular events, in a patient to whom a COX-2 inhibitor is administered.

### DETAILED DESCRIPTION OF THE INVENTION

The interchangeably used terms “cyclooxygenase-2 inhibitor” and “COX-2 inhibitor” according to this invention refer to non-steroidal anti-inflammatory drugs (NSAIDs) which exhibit selectivity for the enzyme cyclooxygenase-2 (COX-2) versus the enzyme cyclooxygenase-1 (COX-1). Therefore the term “COX-2 inhibitor” comprises selective COX-2 inhibitors and specific COX-2 inhibitors. The term “COX-2 inhibitor” includes not only the active form of the corresponding COX-2 inhibitor but also pharmaceutically acceptable salts and prodrugs thereof.

The following compounds including any pharmaceutically acceptable salts or prodrugs thereof are examples of COX-2 inhibitors according to this invention: celecoxib, lumiracoxib, etodolac, meloxicam, nimesulide, rofecoxib, valdecoxib, ABT-963 (Abbott), CS-502 (Sankyo), DRF-4848 (Dr. Reddy's Res. Foundation), E-6087 (Esteve), Etoricoxib (Merck & Co.), GW-406381 (GlaxoSmithKline), NNB-001 (Nobex), NNB-004 (Nobex), NNB-005 (Nobex), Parecoxib Sodium (Pharmacia/Yamanouchi), SVT-2016 (Salvat), Tilmacoxib (Japan Tobacco), UR-8962 (Uriach) and others known to the one skilled in the art.

Preferred COX-2 inhibitors according to this invention are celecoxib, lumiracoxib, etodolac, etoricoxib, meloxicam, nimesulide, rofecoxib and valdecoxib, including any pharmaceutically acceptable salts or prodrugs thereof.

The most preferred COX-2 inhibitors according to this invention are lumiracoxib and meloxicam including any pharmaceutically acceptable salts or prodrugs thereof.

Lumiracoxib is a known specific COX-2 inhibitor. The compound 2-[2-(2-chloro-6-fluorophenylamino)-5-methylphenyl]acetic acid, its synthesis and pharmacological properties including dosage regimens are described for example in Drug of the Future 2002, 27(8), 740-747 and the literature cited therein.

Meloxicam is a known selective COX-2 inhibitor which belongs to the acid enolcarboxamide (oxicam) type of non-steroidal anti-inflammatory drugs (NSAIDs). The compound (4-hydroxy-2-methyl-N-(5-methyl-2-th iazolyl)-2H 1,2-benzothiazine-3-carboxamide 1,1-dioxide) is described in EP 0 002 482 B1 and U.S. Pat. No. 4,233,299.

The pharmaceutically acceptable salts of meloxicam include the sodium salt, potassium salt, ammonium salt, meglumine salt, tris salt, and salts of meloxicam with a basic amino acid. Various salts of meloxicam are described in EP 0 002 482 B1, U.S. Pat. No. 4,233,299 and WO 99/49867.

The term “combined thromboxane receptor antagonist and thromboxane synthase inhibitor” refers to compounds which show a thromboxane-antagonistic activity and an inhibitory activity on thromboxane-synthase including pharmaceutically acceptable salts and prodrugs thereof.

Preferred combined thromboxane receptor antagonist and thromboxane synthase inhibitors according to this invention are compounds, including their pharmaceutically acceptable salts or prodrugs thereof, with thromboxane antagonistic activity measured as IC₅₀ is below 100 nM, more preferably below 50 nM, and which thromboxane synthase inhibitory activity measured as IC₅₀ is below 100 nM, more preferably below 50 nM. Furthermore those combined thromboxane receptor antagonists and thromboxane synthase inhibitors according to this invention are preferred, which exhibit similarly or equally potent activity in both thromboxane receptor blockade and synthase inhibition. Therefore those combined thromboxane receptor antagonists and thromboxane synthase inhibitors according to this invention are preferred, which exhibit a ratio of thromboxane antagonistic activity measured as IC₅₀ versus thromboxane synthase inhibitory activity measured as IC₅₀ smaller or equal 10, even more preferably smaller or equal 5. The thromboxane antagonistic activity and the thromboxane synthase inhibitory activity may be measured for example as described in the EP 547 517 A or by B. Guth et al., Br. J. Clin. Pharmacol. 58 (2004), 40-51.

Preferred combined thromboxane receptor antagonist and thromboxane synthase inhibitors according to this invention are selected from the group consisting of terbogrel, picotamide and derivatives of oxazolecarboxamide-substituted ω-phenyl-ω-(3-pyridyl)-alkenoic acid including compounds as disclosed in EP 0811621, EP 0816361 and U.S. Pat. No. 5,990,308.

The most preferred combined thromboxane receptor antagonist and thromboxane synthase inhibitor according to this invention is terbogrel including pharmaceutically acceptable salts or prodrugs thereof. The advantage of Terbogrel over many other thromboxane receptor antagonists and thromboxane synthase inhibitors is its equally potent thromboxane receptor blockade and synthase inhibition.

Terbogrel is a pyridine derivative of the formula 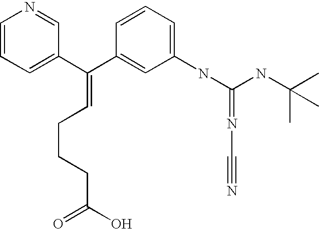
which chemical name is (5E)-6-[3-[[(cyanoamino)[(1,1-dimethylethyl)amino]-methylene]amino]phenyl]-6-(3-pyridinyl)-5-hexenoic acid and wich is described in EP 547517 for example. Its pharmacokinetic and pharmacodynamic properties are described for example by B. Guth et al., Br. J. Clin. Pharmacol. 58 (2004), 40-51.

A preferred pharmaceutical composition according to this invention comprises meloxicam or lumiracoxib or a pharmaceutically acceptable salt thereof and terbogrel or a pharmaceutically acceptable salt thereof.

It will be appreciated that the amount of pharmaceutical composition according to the invention required for use in treatment or prophylaxis will vary not only with the particular compound selected but also with the route of administration, the nature and severity of the condition for which treatment or prophylaxis is required, the age, weight and condition of the patient, concomitant medication and will be ultimately at the discretion of the attendant physician or veterinarian. In general however the active compounds are included in the pharmaceutical composition or dosage form in an amount sufficient to deliver to a patient a therapeutically effective amount of the respective COX-2 inhibitor while the amount of the respective combined thromboxane receptor antagonist and thromboxane synthase inhibitor is sufficient to reduce the risk of adverse events caused by the respective COX-2 inhibitor, in particular to reduce the risk of adverse thromboembolic and/or cardiovascular events.

The therapeutically effective amounts of the respective COX-2 inhibitors to be used in a combination with a thromboxane receptor antagonist and thromboxane synthase inhibitor is usually the same as when administered alone. The therapeutically effective amounts are known to the one skilled in the art and may be adjusted according to well established methods in clinical medicine. The following amounts are given to illustrate the present invention without limiting its scope.

Celecoxib (Celebrex®) is particularly useful when contained in tablets of about 100 to 200 mg. Recommended dosages are typically 100 mg twice per day or 200 mg once per day (see, Bolten, J., Rheumatolog. Suppl., 51:2-7 (May, 1998)). Celecoxib is a preferred COX-2 inhibitor in the compositions and methods of the present invention and should typically be present at 50-500 mg per unit dose.

Rofecoxib (Vioxx®) for oral administration is available in tablets of 12.5, 25 or 50 mg and in an oral suspension containing either 12.5 mg or 25 mg rofecoxib per 5 ml. The recommended initial daily dosage for the management of acute pain is 50 mg. Rofecoxib is a preferred COX-2 inhibitor in the compositions and methods of the present invention and should typically be present at 10-100 mg per unit dose.

A preferred daily dosage of lumiracoxib for an adult patient is in the range of 50 mg to 800 mg, in particular in the range of 100 mg to 400 mg. Therefore a suitable dosage form for oral administration is for example a tablet or capsule comprising 400 mg lumiracoxib for once daily administration or 200 mg lumiracoxib for once or twice daily administration.

Meloxicam (Mobec®) for oral administration is available in tablets of 7.5 mg and 15 mg, in capsules of 5, 7.5, 10 and 15 mg as well as in a suspension containing 7.5 mg meloxicam in 5 ml. In addition meloxicam is available as a solution for injection containing 15 mg meloxicam in 1.5 ml solution. Furthermore suppositories are available containing 7.5 mg or 15 mg of meloxicam. Usually the recommended daily dose of meloxicam for an adult patient is between 1 mg and 30 mg, in particular between 5 and 20 mg.

The combined thromboxane receptor antagonist and thromboxane synthase inhibitor should be present at a level sufficient to prevent or reduce the risk of thromboembolic and/or cardiovascular events. In the case of terbogrel a suitable daily dose for an adult is between 10 and 500 mg, in particular between 50 and 400 mg, even more preferably between 100 and 300 mg. Accordingly when administered twice daily a suitable dose for an adult is between 5 and 250 mg, in particular between 25 and 200 mg. An even more preferred dosage administered twice daily is between 50 and 150 mg, in particular 100 mg.

The desired dose of the pharmaceutical composition according to this invention may conveniently be presented in a single dose or as divided dose administered at appropriate intervals, for example as two, three or more doses per day.

Therefore a preferred pharmaceutical dosage form according to the present invention for oral administration comprises terbogrel or a pharmaceutically acceptable salt thereof and meloxicam or a pharmaceutically acceptable salt thereof wherein the amount of meloxicam or a pharmaceutically acceptable salt thereof is in the range of 1 to 30 mg, even more preferably 5 to 20 mg, and the amount of terbogrel or a pharmaceutically acceptable salt thereof is in the range of 10 to 500 mg, even more preferably 50 to 400 mg.

Another preferred pharmaceutical dosage form according to the present invention for oral administration comprises terbogrel or a pharmaceutically acceptable salt thereof and lumiracoxib or a pharmaceutically acceptable salt thereof wherein the amount of lumiracoxib or a pharmaceutically acceptable salt thereof is in the range of 50 to 800 mg, even more preferably 100 to 400 mg, and the amount of terbogrel or a pharmaceutically acceptable salt thereof is in the range of 10 to 500 mg, even more preferably 50 to 400 mg.

The compositions according to this invention preferably also comprise at least one pharmaceutically acceptable carrier.

The pharmaceutical composition according to the present invention is conveniently administered in a dosage form; for example containing 5 to 3000 mg, conveniently 5 to 1000 mg of active ingredient(s) per unit dosage form.

The combined thromboxane receptor antagonist and thromboxane synthase inhibitor and the COX-2 inhibitor may be formulated as a single pharmaceutical dosage form or as two separate dosage forms. In case of separate dosage forms, these may be administered either simultaneously or sequentially.

Therefore the present invention also relates to a multiple dosage form, preferably a kit of parts, which is useful in combination therapy to flexibly suit the individual therapeutic needs of the patient.

A preferred kit of parts comprises
(a) a first containment containing a pharmaceutical composition comprising a combined thromboxane receptor antagonist and thromboxane synthase inhibitor and at least one pharmaceutically acceptable carrier, and
(b) a second containment containing a pharmaceutical composition comprising a COX-2 inhibitor and at least one pharmaceutically acceptable carrier.

An even more preferred kit of parts comprises
(a) a first containment containing a pharmaceutical composition comprising a therapeutically effective amount of terbogrel or a pharmaceutically acceptable salt thereof and at least one pharmaceutically acceptable carrier, and
(b) a second containment containing a pharmaceutical composition comprising a therapeutically effective amount of lumiracoxib or meloxicam or a pharmaceutically acceptable salt thereof and at least one pharmaceutically acceptable carrier.

A preferred manufacture comprises a combined thromboxane receptor antagonist and thromboxane synthase inhibitor and a COX-2 inhibitor for use in combination or alternation.

Pharmaceutical dosage forms include those suitable for oral, rectal, nasal, topical (including buccal and sublingual), transdermal, vaginal or parenteral (including intramuscular, sub-cutaneous and intravenous) administration in liquid or solid form or in a form suitable for administration by inhalation or insufflation. The formulations may, where appropriate, be conveniently presented in discrete dosage units and may be prepared by any of the methods well known in the art of pharmacy. All methods include the step of bringing into association the active compound(s) with liquid carriers or finely divided solid carriers or both and then, if necessary, shaping the product into the desired formulation.

The pharmaceutical dosage forms described in the present invention comprise tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

The pharmaceutical acceptable carrier(s) must be “acceptable” in the sense of being compatible with the other ingredients of the formulation and not deleterious to the recipient thereof.

Pharmaceutical compositions suitable for oral administration may conveniently be presented as discrete units such as capsules, including soft gelatin capsules, cachets or tablets each containing a predetermined amount of the active ingredient(s); as a powder or granules; as a solution, a suspension or as an emulsion, for example as syrups, elixirs or self-emulsifying delivery systems (SEDDS). The active ingredient(s) may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical composition according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient(s) may be in powder form, obtained by aseptic isolation of sterile solid or by lyophilisation from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in moulds.

When desired the above described compositions and dosage forms may be adapted to give sustained release of the active ingredient(s).

The pharmaceutical dosage forms and compositions may be prepared using regular methods, and, in addition to the aforementioned components, any additives in common use may be used upon preparation of these formulations, if necessary. In addition, preparations formed into microparticles such as microcapsules, nanocapsules, microspheres, nanospheres, may also be included in the aforementioned formulations.

In addition, further components of the oral pharmaceutical dosage form and the formulation of all ingredients are preferably chosen in view of the desired mechanical, chemical and biological stability, release rate, masking of the taste, visual appearance, etc.

For example, the pharmaceutically active substances according to this invention, can be dispensed in separate granules, multi-layer granules, multi-layer tablets or dry coated tablets, tablets of separated granules, microcapsules, etc. Coating preparations such as sugarcoated tablets, film coating tablets, coating granule, can be used as well as chewable tablets, oral fast dispersing tablets, matrix tablets, matrix granules, effervescent tablets, dusting powder, solid solutions, etc. These methods can also be combined. Moreover, the properties of the inventive pharmaceutical dosage form such as stability, release, continuance, disintegration, distinglation, dissolution, concealment of taste, improvement in usage etc. can be regulated by the addition of additives known in the art.

These dosage forms described in this invention may be prepared using regular methods by adding generally available pharmaceutical additives such as excipients, binders, disintegrators, lubricants, coating agents, sugar coating agents, plasticizers, antifoaming agents, polish, foaming agents, antistatic agents, desiccant, surfactant, solubilizer, buffer agents, resolvents, solubilizing agents, solvents, diluents, stabilizers, emulsifying agents, suspending agents, dispersing agents, isotonizing agents, adsorbents, reducing agents, antioxidant, wetting agents, wet modifier, filler, extender, adhesives, viscous agent, softeners, pH modifiers, antiseptics, preservatives, sweetening agents, flavoring agents, and coloring matters to the pharmacologically active compounds.

Examples of pharmaceutically acceptable carriers are magnesium stearate, chalk, starch, lactose, wax, gum or gelatin. Carriers which are suited to achieve a sustained release, for example natural or synthetic polymers or liposomes, are known to the one skilled in the art. Pharmaceutically acceptable carriers also comprise liquid carriers and diluents, for example water, alcohol, glycerine or oil, which serve as a base for liquid formulations, such as solutions, suspensions or emulsions.

The compositions, combinations, dosage forms, kit of parts and manufacture according to this invention are advantageous in view of their antiphlogistic activity and are particularly useful for the treatment of cyclooxygenase dependent disorders in a patient, including inflammation, pain of inflammation and for treating rheumatic diseases, in particular pyresis, osteoarthritis, rheumatoid arthritis, migraine headache, neurodegenerative diseases (such as multiple sclerosis), Alzheimer's disease, osteoporosis, asthma, lupus and psoriasis.

The compositions, combinations, dosage forms, kit of parts and manufacture according to the invention are therefore suitable for treating all peracute, acute, subacute, chronic and recurring inflammation, particularly for treating the symptoms of acute episodes of intermittent or chronic activated arthrosis as well as for long-term symptomatic treatment of rheumatoid arthritis (chronic polyarthritis) and for the symptomatic treatment of ankylosing spondylitis (Bechterew's disease).

The compositions, combinations, dosage forms, kit of parts and manufacture according to the invention are also suitable for treating acute pain, such as for example toothache after tooth extractions, post-traumatic and postoperative pain, headache, acute sciatica, acute back pain, tendonitis, cervicobrachial syndrome and tennis elbow as well as for the treatment of persistent pain, such as for example backache or pain caused by tumours.

The compositions, combinations, dosage forms, kit of parts and manufacture according to the invention are further useful for the treatment of neoplasia particularly neoplasia that produce prostaglandins or express cyclooxygenase, including both benign and cancerous tumors, growths and polyps. Neoplasias which (frequently) produce prostaglandins comprise for example malignant brain tumours, bone cancer, epithelial cell neoplasia such as basal cell carcinoma, adenocarcinoma, cancers of the gastrointestinal tract such as lip cancer, mouth cancer, oesophageal cancer, cancer of the small intestine and stomach cancer, large bowel cancer, liver cancer, bladder cancer, pancreatic cancer, ovarian cancer, cancer of the womb, lung cancer, breast cancer and skin cancer, prostate cancer, kidney cell carcinoma and other known types of cancer which affect the epithelial cells in the body.

The dosage ranges with respect to the combined thromboxane receptor antagonist and thromboxane synthase inhibitor and with respect to the COX-2 inhibitor which are preferred in the methods of treatment and in the uses according to this invention are as specified above.

The term “treatment” as used herein is to be understood as including both therapeutic and prophylactic modes of therapy. For example in relation to the treatment of neoplasia, therapy to prevent the onset of clinically or preclinically evident neoplasia, or for the prevention of initiation of malignant cells or to arrest or reverse the progression of premalignant to malignant cells, as well as the prevention or inhibition of neoplasia growth or metastasis.

The compounds of the invention have activity in the above indications while substantially avoiding or preventing adverse thromboembolic and/or cardiovascular events.

Patients to be treated according to this invention are preferably mammals, especially humans, but also animals, particularly domestic pets or farm animals, such as pigs, horses, cattle, sheeps, dogs or cats.

The patients to be treated are especially those individuals that in addition to the one or more cyclooxygenase dependent disorders and indications listed above can be characterized by one or more of the following conditions, needs or risks:
a need for a cardiovascular protective treatment;
a risk for cardiovascular events, such as angina, prior myocardial infarction or cardiac revascularization, atherosclerotic heart disease;
hypertensive heart disease;
congestive heart failure and other ischemic heart disease;
established coronary artery disease;
coronary conduction disorders, cardiac arhythmias;
transient ischemic attacks, cerebrovascular accidents;
peripheral artery disease;
familial history of, or genetically predisposed to, thromboembolic disorders;
patients with altered prostacyclin/thromboxane A2 homeostasis or higher than normal thromboxane A2 levels;
patients with diabetes and/or rheumatoid arthritis;
hypertension;
a renal insufficiency or condition, or a risk for renal failure.

An example of a method of treating cyclooxygenase dependent disorders in an adult patient according to this invention comprises administering
7.5 mg meloxicam once or twice daily and 100 mg terbogrel once or twice daily; or
7.5 or 15 mg meloxicam once daily and 200 mg terbogrel once daily or 100 mg terbogrel twice daily.

A suitable pharmaceutical dosage form for once or twice daily administration comprises for example 7.5 mg meloxicam and 100 mg terbogrel. For once daily administration a suitable dosage form comprises 7.5 or 15 mg meloxicam and 200 mg terbogrel for example.

A further example of a method of treating cyclooxygenase dependent disorders in an adult patient according to this invention comprises administering
400 mg lumiracoxib once daily and 200 mg terbogrel once daily or 100 mg terbogrel twice dialy; or
200 mg lumiracoxib once or twice daily and 100 mg terbogrel once or twice daily.

A suitable pharmaceutical dosage form for once daily administration comprises for example 400 mg lumiracoxib and 200 mg terbogrel. Another example for once or twice daily administration is a pharmaceutical dosage form comprising 200 mg lumiracoxib and 100 mg terbogrel.

An example of a kit of parts or manufacture according to this invention comprises dosage forms such as tablets or capsules comprising meloxicam in a therapeutic dose such as for example 7.5 mg and dosage forms such as tablets or capsules comprising terbogrel in a dose sufficient to prevent or reduce the risk of thromboembolic and/or cardiovascular events such as for example 100 mg. An example of a suitable administration scheme for an adult patient involves administering the dosage form comprising meloxicam once daily and administering the dosage form comprising terbogrel once or twice daily.

A further example of a kit of parts or manufacture according to this invention comprises dosage forms such as tablets or capsules comprising lumiracoxib in a therapeutic dose such as for example 400 mg and dosage forms such as tablets or capsules comprising terbogrel in a dose sufficient to prevent or reduce the risk of thromboembolic and/or cardiovascular events such as for example 100 mg. An example of a suitable administration scheme for an adult patient involves administering the dosage form comprising lumiracoxib once daily and administering the dosage form comprising terbogrel once or twice daily.

What is claimed is:

1. A pharmaceutical composition comprising a first active ingredient, wherein the first active ingredient is a combined thromboxane receptor antagonist and thromboxane synthase inhibitor, and a second active ingredient, wherein the second active ingredient is a COX-2 inhibitor.

2. The pharmaceutical composition according to claim 1, wherein the combined thromboxane receptor antagonist and thromboxane synthase inhibitor is terbogrel, or a pharmaceutically acceptable salt or prodrug thereof.

3. The pharmaceutical composition according to claim 1, wherein the COX-2 inhibitor is selected from the group consisting of celecoxib, lumiracoxib, etodolac, etoricoxib, meloxicam, nimesulide, rofecoxib and valdecoxib, or a pharmaceutically acceptable salt or prodrug thereof.

4. The pharmaceutical composition according to claim 3, wherein the COX-2 inhibitor is meloxicam or lumiracoxib, or a pharmaceutically acceptable salt or prodrug thereof.

5. The pharmaceutical composition according to claim 1, further comprising at least one pharmaceutically acceptable carrier or excipient.

6. A pharmaceutical dosage form comprising the pharmaceutical composition according to claim 1.

7. The pharmaceutical dosage form according to claim 6, for oral administration once or twice daily, comprising terbogrel, or a pharmaceutically acceptable salt thereof, and meloxicam, or a pharmaceutically acceptable salt thereof, wherein the amount of meloxicam, or a pharmaceutically acceptable salt thereof, is in the range of 1 to 30 mg and the amount of terbogrel, or a pharmaceutically acceptable salt thereof, is in the range of 10 to 500 mg.

8. The pharmaceutical dosage form according to claim 6 for oral administration, once or twice daily, comprising terbogrel, or a pharmaceutically acceptable salt thereof, and lumiracoxib, or a pharmaceutically acceptable salt thereof, wherein the amount of lumiracoxib, or a pharmaceutically acceptable salt thereof, is in the range of 50 to 800 mg and the amount of terbogrel, or a pharmaceutically acceptable salt thereof, is in the range of 10 to 500 mg.

9. A method of treating a cyclooxygenase dependent disorder comprising administering to an individual in need of such treatment the pharmaceutical dosage form of claim 6.

10. The method according to claim 9, wherein the disorder is one or more of the disorders selected from the group consisting of inflammation, pain, rheumatic diseases and neoplasia.

11. A method of minimizing the incidence of adverse thromboembolic or cardiovascular events in an individual to whom a COX-2 inhibitor has been administered comprising administering to said individual a pharmaceutical composition comprising a combined thromboxane receptor antagonist and thromboxane synthase inhibitor.

12. A method of treating a cyclooxygenase dependent disorder, comprising administering to an individual in need of such treatment a pharmaceutical composition comprising a first active ingredient, wherein the first active ingredient is a combined thromboxane receptor antagonist and thromboxane synthase inhibitor, and a second active ingredient, wherein the second active ingredient is a COX-2 inhibitor.

13. The method according to claim 12, wherein the disorder is one or more of the disorders selected from the group consisting of inflammation, pain, rheumatic diseases and neoplasia.

14. The method according to claim 12, wherein the combined thromboxane receptor antagonist and thromboxane synthase inhibitor and the COX-2 inhibitor are formulated as a single pharmaceutical dosage form.

15. The method according to claim 12, wherein the combined thromboxane receptor antagonist and thromboxane synthase inhibitor and the COX-2 inhibitor are formulated as two separate pharmaceutical dosage forms.

16. The method according to claim 12, wherein the COX-2 inhibitor is selected from the group consisting of celecoxib, lumiracoxib, etodolac, etoricoxib, meloxicam, nimesulide, rofecoxib and valdecoxib, or a pharmaceutically acceptable salt or prodrug thereof.

17. The method according to claim 12, wherein the combined thromboxane receptor antagonist and thromboxane synthase inhibitor is terbogrel, or a pharmaceutically acceptable salt or prodrug thereof.

18. The method according to claim 12, wherein the cyclooxygenase dependent disorder, is one or more of the disorders selected from the group consisting of pyresis, osteoarthritis, rheumatoid arthritis, migraine headache, neurodegenerative diseases, Alzheimer's disease, osteoporosis, asthma, lupus, psoriasis, acute pain and persistent pain.

19. The method according to claim 12, wherein the individual is characterized by one or more of the following conditions, needs or risks:
a need for a cardiovascular protective treatment;
a risk for cardiovascular events, such as angina, prior myocardial infarction or cardiac revascularization, atherosclerotic heart disease;
hypertensive heart disease;
congestive heart failure and other ischemic heart disease;
established coronary artery disease;
coronary conduction disorders, cardiac arhythmias;
transient ischemic attacks, cerebrovascular accidents;
peripheral artery disease;
familial history of, or genetically predisposed to, thromboembolic disorders;
individauls with altered prostacyclin/thromboxane A2 homeostasis or higher than normal thromboxane A2 levels;
individauls with diabetes and/or rheumatoid arthritis;
hypertension; or
a renal insufficiency or condition, or a risk for renal failure.

20. A kit comprising
(a) a first containment containing a pharmaceutical composition comprising a combined thromboxane receptor antagonist and thromboxane synthase inhibitor and at least one pharmaceutically acceptable carrier, and
(b) a second containment containing a pharmaceutical composition comprising a COX-2 inhibitor and at least one pharmaceutically acceptable carrier.
